(19) United States
(12) Patent Application Publication
(10) Pub. No.: US 2024/0010596 A1
(43) Pub. Date: Jan. 11, 2024

(54) INTEGRATED CARBOXYLIC ACID PRODUCTION FROM SYNTHESIS GAS

(71) Applicant: Rohm and Haas Company, Collegeville, PA (US)

(72) Inventors: Joseph F. Dewilde, King of Prussia, PA (US); Kirk W. Limbach, Dresher, PA (US); Reetam Chakrabarti, Phoenixville, PA (US)

(21) Appl. No.: 18/253,605

(22) PCT Filed: Nov. 17, 2021
(86) PCT No.: PCT/US2021/059594

§ 371 Date: May 19, 2023

Related U.S. Application Data
(63) Continuation of application No. 63/119,736, filed on Dec. 1, 2020.

(51) Int. Cl. C07C 51/225; C07C 51/64

(52) U.S. Cl. CPC C07C 51/225; C07C 51/64; C07C 2521/06; C07C 2523/08; C07C 2529/85

(57) ABSTRACT

A process for preparing C₂ to C₄ carboxylic acids from synthesis gas includes introducing a feed stream comprising hydrogen gas and a carbon-containing gas comprising carbon monoxide into a reaction zone of a first reactor, converting the feed stream into an intermediate stream comprising C₂ to C₄ hydrocarbons in the reaction zone in the presence of a first catalyst, wherein the intermediate stream further comprises carbon dioxide and wherein the first catalyst is a composite catalyst comprising a metal oxide catalyst component and a microporous catalyst component, and converting the intermediate stream into a product stream comprising C₂ to C₄ carboxylic acids in the presence of a second catalyst in a second reactor. The second reactor can be configured for olefin oxidation or paraffin oxidation.

### BACKGROUND

### Field

The present specification generally relates to processes that efficiently convert various carbon-containing streams to carboxylic acids via C₂ to C₄ hydrocarbons.

### Technical Background

For a number of industrial applications, hydrocarbons are used, or are starting materials used, to produce plastics, fuels, and various downstream chemicals. C₂ to C₄ hydrocarbons are particularly useful in downstream applications, such as, for example, preparing carboxylic acids, such as acrylic acid. Acrylic acid is a high-value chemical intermediate for the production of acrylate products, including superabsorbent polymers and acrylate esters that are used in paints and coatings.

A variety of processes for producing lower hydrocarbons has been developed, including petroleum cracking and various synthetic processes. Synthesis gas, known as syngas, a combination of carbon monoxide and hydrogen gas, represents a flexible intermediate that can be obtained from the gasification of biomass, waste, or conventional fuels.

Synthetic processes for converting syngas to lower hydrocarbons, are known. The Fischer-Tropsch process has been used to convert syngas to a mixture of olefins along with longer chain paraffins. The Fischer-Tropsch process produces a broad product distribution and selectivity of lower olefins is typically relatively limited. To increase selectivity to lower olefins, variations of the Fischer-Tropsch process have been developed, such as the process disclosed in WO 2019/089206.

However, there are at least two azeotropes that encumber the separation in typical syngas to olefin plants, including ethylene/carbon dioxide and ethane/carbon dioxide. See Nagahama et al., Journal of Chemical Engineering of Japan, 7, 5, 1974, pp. 323-328. Separating the carbon dioxide from the olefins is a costly process. While extractive distillation can help break one or the other of these azeotropes, the presence of both azeotropes encourages the use of a non-distillation separation, such as amine scrubbing, of CO₂ in syngas to olefin operations.

Some of these processes include co-feeding CO₂ to the process to reduce the net CO₂ selectivity, determined by the CO₂ in the product stream less the total CO₂ in the feed stream, which may be negative. However, this approach typically leads to reduced productivity of the desired C₂ to C₄ hydrocarbons.

Other process, such as that disclosed in U.S. Pat. No. 10,513,471, use a special catalyst to minimize formation of CO₂ in a two-reactor process that converts syngas to C₂ to C₅ hydrocarbons.

U.S. Pat. No. 10,676,419 discloses a two-stage Fischer-Tropsch process for converting syngas to acetic acid, acrylic acid, and/or propylene. In a first stage, syngas is contacted with a first Fischer-Tropsch catalyst to produce a first product stream comprising C₂ and C₃ olefins and/or C₂ and C₃ paraffins, and the first product stream is then contacted with oxygen gas a second catalyst to produce acrylic acid and acetic acid.

Accordingly, a need exists for processes and systems in which carboxylic acids can be produced from syngas efficiently and with high yield.

### SUMMARY

One aspect of the present invention relates to a process comprising introducing a feed stream comprising hydrogen gas and a carbon-containing gas comprising carbon monoxide into a reaction zone of a first reactor, converting the feed stream into an intermediate stream comprising C₂ to C₄ hydrocarbons in the reaction zone in the presence of a first catalyst, wherein the first product stream further comprises carbon dioxide and wherein the first catalyst is a composite catalyst comprising a metal oxide catalyst component and a microporous catalyst component, and converting the intermediate stream into a product stream comprising C₂ to C₄ carboxylic acids in the presence of a second catalyst in a second reactor.

Additional features and advantages will be set forth in the detailed description which follows, and in part will be readily apparent to those skilled in the art from that description or recognized by practicing the embodiments described herein, including the detailed description which follows and the claims.

It is to be understood that both the foregoing general description and the following detailed description describe various embodiments and are intended to provide an overview or framework for understanding the nature and character of the claimed subject matter.

### DETAILED DESCRIPTION

As used herein, it is noted that “synthesis gas” and “syngas” are utilized herein to represent a mixture comprising primarily hydrogen, carbon monoxide, and very often some carbon dioxide.

Reference will now be made in detail to embodiments of processes utilizing syngas to prepare C2 to C4 hydrocarbons and further to C2 to C4 carboxylic acids.

In general, in syngas to hydrocarbon processes, it is desirable to achieve a high productivity of the desired C2 to C4 hydrocarbons, while simultaneously reducing the net selectivity of CO2. A known method to reduce the net selectivity of CO2 is by co-feeding CO2. However, by co-feeding additional CO2 to reduce the net selectivity of CO2, this also results in a decreased productivity of the desired C2 to C4 hydrocarbons. However, the present inventors have recognized that by directly subjecting a product stream comprising C2 to C4 hydrocarbons and CO2 to an olefin oxidation process or a paraffin oxidation process to produce C2 to C4 carboxylic acids, such as acrylic acid, the CO2 can pass as an inert. Further, any unreacted or generated CO and CO2 can be recycled back to the syngas feed stream for more complete carbon utilization.

By eliminating the need for breaking any azeotropes formed with CO2, such as, for example, ethylene/CO2 or ethane/CO2, significant savings can be achieved in capital and operating costs, in addition to the more complete carbon utilization that can be achieved when the CO2 is recycled back to the syngas feed stream.

In the process of the present invention, a feed stream comprising hydrogen gas and a carbon-containing gas comprising carbon monoxide is introduced into a reaction zone of a first reactor. Preferably, the feed stream comprises syngas. The syngas may comprise hydrogen and carbon monoxide, which may optionally be supplemented with carbon dioxide depending on the level of carbon dioxide, if any, present in the syngas.

The feed stream is converted into an intermediate stream in the reaction zone in the presence of a first catalyst. The first catalyst is a composite or hybrid catalyst comprising a metal oxide component and a microporous catalyst component. The intermediate stream, which comprises carbon dioxide, is then converted into a product stream comprising C2 to C4 carboxylic acids in the presence of a second catalyst in a second reactor. The product stream may further comprise C2 to C4 ketones.

The intermediate stream comprising C2 to C4 hydrocarbons preferably comprises C2 to C4 olefins and C2 to C4 paraffins. The second reactor can be configured for olefin oxidation or paraffin oxidation.

When the second reactor is configured for olefin oxidation, the C2 to C4 olefins in the intermediate stream are preferentially converted to C2 to C4 carboxylic acids. The C2 to C4 carboxylic acids can be separated from the product stream, leaving paraffins and carbon dioxide in the remainder of the product stream. Preferably, the paraffins are separated and the carbon dioxide is recycled to the feed stream.

When the second reactor is configured for paraffin oxidation, the C2 to C4 paraffins and potentially any co-produced olefins are preferentially converted to C2 to C4 carboxylic acids. The C2 to C4 carboxylic acids can be separated from the product stream. Preferably, any unreacted paraffins and carbon dioxide are recycled to the feed stream.

The hydrogen may present in the feed stream in an amount of from 10.0 vol % to 90.0 vol % H2, such as from 20.0 vol % to 80.0 vol % H2 or from 30.0 vol % to 70.0 vol % H2 based on the total volume of the feed stream.

The carbon dioxide may be present in the feed stream in an amount of from 0 vol % to 20.0 vol % CO2 relative to the total volume of the feed stream. Any carbon dioxide present in the feed stream may be present in the syngas or recycled back to the first feed stream from the second product stream. Fresh carbon dioxide, e.g., a carbon dioxide co-feed, does not need to be added to the first feed stream as the process may use recycled carbon dioxide generated in the first or second reactors.

The first catalyst comprises a metal oxide catalyst component and a microporous catalyst component, such as, for example, a zeolite component.

The metal oxide catalyst component may be a bulk catalyst or a supported catalyst and may be made by any suitable method, such as co-precipitation, impregnation, or the like. The metal oxide catalyst may comprise, for example, zinc oxide, chromium oxide, copper oxide, aluminum oxide, gallium oxide, zirconium oxide, and combinations thereof. It should be understood that any metal in the metal oxide component mixture can be present in a variety of oxidation states. It should also be understood that the designation of a specific oxide (e.g. Ga2O3), does not necessarily preclude the presence of an additional or different oxide of the given metal(s).

Preferably, when the second reactor is configured for olefin oxidation, the metal oxide catalyst component comprises gallium oxide and zirconium oxide. The zirconium oxide may be phase pure zirconia. As used herein, “phase pure zirconia” means ZrO2 to which no other materials have intentionally been added during formation. Thus, “phase pure zirconia” includes zirconia with small amounts of components other than zirconium (including oxides other than zirconia) that are unintentionally present in the zirconia as a natural part of the zirconia formation process, such as, for example, hafnium.

The composition of the metal oxide catalyst component is designated by a weight percentage of the gallium metal to the pure zirconia (accounting for ZrO2 stoichiometry). For example, the composition of the metal oxide catalyst component can be designated by weight of gallium per 100 grams (g) of zirconia. Preferably, the metal oxide catalyst component comprises from greater than 0.0 g gallium to 30.0 g gallium per 100 g of zirconia, such as 5.0 g gallium to 30.0 g gallium per 100 g of zirconia, 10.0 g gallium to 30.0 g gallium per 100 g of zirconia, 15.0 g gallium to 30.0 g gallium per 100 g of zirconia, 20.0 g gallium to 30.0 g gallium per 100 g of zirconia, or 25.0 g gallium to 30.0 g gallium per 100 g of zirconia.

Preferably, when the second reactor is configured for paraffin oxidation, the metal oxide catalyst component of the first catalyst is selected from gallium oxide, zinc-chromium mixed oxides, or copper-zinc-aluminum mixed oxides.

The microporous catalyst component is preferably selected from molecular sieves having 8-MR pore openings and having a framework type selected from the group consisting of the following framework types CHA, AEI, AFX, ERI, LTA, UFI, RTH, EDI, GIS, MER, RHO, and combinations thereof, the framework types corresponding to the naming convention of the International Zeolite Association. It should be understood both aluminosilicate and silicoaluminophosphate frameworks may be used. The microporous catalyst component may include tetrahedral aluminosilicates, ALPOs (such as, for example, tetrahedral aluminophosphates), SAPOs (such as, for example, tetrahedral silicoaluminophosphates), and silica-only based tectosilicates. Preferably, the microporous catalyst component is a silicoaluminophosphate having a Chabazite (CHA) framework type. Examples of these may include, but are not necessarily limited to: CHA framework types selected from SAPO-34 and SSZ-13; and AEI framework types such as SAPO-18. Combinations of microporous catalyst components having any of the above framework types may also be employed. It should be understood that the microporous catalyst component may have different membered ring pore opening depending on the desired product. For instance, microporous catalyst component having 8-MR to 12-MR pore openings could be used depending on the desired product.

The metal oxide catalyst component and the microporous catalyst component of the first catalyst may be mixed together by any suitable means, such as, for example, by physical mixing-such as shaking, stirring, or other agitation. The metal oxide catalyst component may, in embodiments, comprise from 1.0 wt % to 99.0 wt % of the first catalyst, such as from 5.0 wt % to 95.0 wt % of the hybrid catalyst, such as from 10.0 wt % to 90.0 wt %, from 15.0 wt % to 85.0 wt %, from 20.0 wt % to 80.0 wt %, from 25.0 wt % to 75.0 wt %, from 30.0 wt % to 70.0 wt %, from 35.0 wt % to 65.0 wt %, from 40.0 wt % to 60.0 wt %, or from 45.0 wt % to 55.0 wt % based on the total weight of the first catalyst.

The metal oxide catalyst component may be reduced within the reactor prior to exposure to the first feed stream by exposing the metal oxide catalyst component to conventional reducing gases. Alternatively, the metal oxide catalyst component may be reduced within the reactor upon exposure to reducing gases in the feed stream such as H2 and CO.

The reaction conditions within the reaction zone of the first reactor will now be described. The feed stream contacted with the first catalyst in the reaction zone of the first reactor under reaction conditions sufficient to form a first product stream comprising C2 to C4 hydrocarbons. The first product stream may further comprise higher hydrocarbons, i.e., C5 or higher hydrocarbons. Preferably, the first product stream comprises primarily C2 to C4 hydrocarbons. The reaction conditions comprise a temperature within the reaction zone ranging, for example, from 300° C. to 500° C., such as from 380° C. to 450° C., from 380° C. to 440° C., from 380° C. to 430° C., from 380° C. to 420° C., from 380° C. to 410° C., from 380° C. to 400° C., or from 380° C. to 390° C.

The reaction conditions also include, for example, a pressure inside the reaction zone of at least 20 bar (20,000 kilopascals (kPa)), such as at least 25 bar (25,000 kPa), at least 30 bar (30,000 kPa), at least 35 bar (35.00 kPa), at least 40 bar (40,000 kPa), at least 45 bar (45,000 kPa), at least 50 bar (50,000 kPa), at least 55 bar (55,000 kPa), at least 60 bar (60,000 kPa), at least 65 bar (65,000 kPa), or at least 70 bar (70,000 kPa).

The reaction conditions also include, for example, a gas hourly space velocity inside the reaction zone 101 of at least 2500 hr-1, such as at least 3000 hr-1, such as at least 3600 hr-1, such as at least 4200 hr-1, such as at least 4800 hr-1, such as at least 5400 hr-1, such as at least 6000 hr-1, such as at least 6600 hr-1, or such as at least 7200 hr-1.

The intermediate stream comprises C2 to C4 hydrocarbons and further comprises carbon dioxide. The carbon dioxide present in the intermediate stream is not removed from the intermediate stream. The intermediate stream may also contain unreacted carbon monoxide and hydrogen. Preferably, the carbon monoxide, is removed from the intermediate stream and recycled to the feed stream. Any hydrogen present may also be recycled to the feed stream.

C2 to C4 hydrocarbons in the intermediate stream are then converted to C2 to C4 carboxylic acids in the presence of a second catalyst and with the addition of oxygen or an oxygen-containing gas such as air.

When the second reactor is configured for olefin oxidation, the C2 to C4 olefins in the intermediate stream are preferentially converted to C2 to C4 carboxylic acids in the presence of a second catalyst selected for catalyzing the conversion of the C2 to C4 olefins. The second catalyst may be selected from any catalysts known in the art for converting olefins to carboxylic acids. Examples include, but are not limited to, palladium salts, copper salts, as well as metal oxides of bismuth, vanadium, molybdenum and combinations thereof.

When the second reactor is configured for paraffin oxidation, the C2 to C4 paraffins and olefins in the intermediate stream are preferentially converted to C2 to C4 carboxylic acids in the presence of a second catalysts selected for catalyzing the conversion of the C2 to C4 paraffins. The second catalyst may be selected from any catalysts known in the art for converting paraffins to carboxylic acids. Examples include, but are not limited to, ceria, as well as metal oxides of bismuth, vanadium, molybdenum, tellurium and combinations thereof.

In the second reactor, the reaction conditions comprise a temperature ranging from 300° C. to 450° C., preferably from 350° C. to 400° C. Either air or purified oxygen can be fed to the second reactor. Preferably, purified oxygen is fed to the second reactor to avoid subsequent removal of nitrogen when air is used.

For oxidation of olefins, the second reactor may comprise two reactors operating at two different temperatures, including a reactor for producing an aldehyde, and a subsequent reactor for producing the product carboxylic acid from the aldehyde. The catalysts may be the same or different and are selected from the second catalysts described above.

For oxidation of paraffins, multiple reactors may also be used. However, the carboxylic acids may be removed using an interstage separation between the reactors to minimize over oxidation of the desired product to carbon dioxide or carbon monoxide.

The C2 to C4 carboxylic acids produced by the oxidation reaction may be separated from the product stream. Any ketones that are produced during the oxidation reaction may be removed with the carboxylic acids for further separation or use. In the case where the C2 to C4 hydrocarbons in the intermediate stream are subjected to olefin oxidation, any paraffins in the product stream may be separated from the product stream and the carbon dioxide and any carbon monoxide present may be recycled to the feed stream to further optimize carbon usage. When the second reactor is confided for paraffin oxidation, any paraffins present in the product stream may be recycled to the feed stream with the carbon dioxide and any carbon monoxide present.

It will be apparent to those skilled in the art that various modifications and variations can be made to the embodiments described herein without departing from the spirit and scope of the claimed subject matter. Thus, it is intended that the specification cover the modifications and variations of the various embodiments described herein provided such modification and variations come within the scope of the appended claims and their equivalents.

1. A process comprising:
introducing a feed stream comprising hydrogen gas and a carbon-containing gas comprising carbon monoxide and carbon dioxide into a reaction zone of a first reactor;
converting the feed stream into an intermediate stream comprising C₂ to C₄ hydrocarbons in the reaction zone in the presence of a first catalyst, wherein the intermediate stream further comprises carbon dioxide and wherein the first catalyst is a composite catalyst comprising a metal oxide catalyst component and a microporous catalyst component; and
converting the intermediate stream into a product stream comprising C₂ to C₄ carboxylic acids in the presence of a second catalyst in a second reactor.

2. The process of claim 1, wherein the intermediate stream comprising C₂ to C₄ hydrocarbons comprises C₂ to C₄ olefins, C₂ to C₄ paraffins, carbon dioxide, and unreacted carbon monoxide and hydrogen.

3. The process of claim 2, wherein the second reactor converts the C₂ to C₄ olefins in the intermediate stream to C₂ to C₄ carboxylic acids.

4. The process of claim 3, further comprising separating the C₂ to C₄ carboxylic acids and C₂ to C₄ paraffins from the product stream and recycling carbon dioxide and carbon monoxide to the feed stream.

5. The process of claim 1, wherein the metal oxide catalyst component comprises gallium oxide, zinc oxide, chromium oxide, zirconium oxide, or combinations thereof.

6. The process of claim 5, wherein the metal oxide catalyst component comprises gallium oxide and phase pure zirconium oxide.

7. The process of claim 2, wherein the second reactor converts the C₂ to C₄ paraffins in the intermediate stream to the product stream comprising C₂ to C₄ carboxylic acids.

8. The process of claim 7, further comprising separating the C₂ to C₄ carboxylic acids from the product stream and recycling carbon dioxide, carbon monoxide, and unreacted C₂ to C₄ paraffins to the feed stream.

9. The process of claim 1, wherein the metal oxide catalyst component comprises gallium oxide.

10. The process of claim 1, wherein unreacted hydrogen is removed from the intermediate stream prior to converting the intermediate stream into the product stream.

11. The process of claim 1, wherein the microporous catalyst component is a molecular sieve having 8-MR pore openings.

12. The process of claim 1, wherein the microporous catalyst component is a molecular sieve having a Chabazite (CHA) framework.

13. The process of claim 1, wherein the microporous catalyst component comprises SAPO-34.

14. The process of claim 1, wherein the microporous catalyst component comprises SSZ-13.
